(19) United States
(12) Patent Application Publication
(10) Pub. No.: US 2015/0192553 A1
(43) Pub. Date: Jul. 9, 2015

(54) APPARATUS FOR TESTING PERFORMANCE OF CARBON DIOXIDE SORBENT

(71) Applicant: KOREA TESTING LABORATORY (KTL), Seoul (KR)

(72) Inventors: Kwang Koo Kim, Gwangmyeong-si (KR); Kil Hwan Hong, Gwangmyeong-si (KR); Min Chul Shin, Seoul (KR); Jin Sun Cha, Seoul (KR); Mi Jin Jeon, Seoul (KR)

(21) Appl. No.: 14/418,650

(22) PCT Filed: May 16, 2014
(86) PCT No.: PCT/KR2014/004405

§ 371 Date: Jan. 30, 2015

(30) Foreign Application Priority Data
Jan. 9, 2014 (KR) 10-2014-0003040

(51) Int. Cl. G01N 33/00

(52) U.S. Cl. CPC G01N 33/004

(57) ABSTRACT

Provided is an apparatus for testing the performance of carbon dioxide sorbents, the apparatus including: a reactor accommodating carbon dioxide sorbents therein; an injection part coupled with the reactor and injecting a gas mixture containing carbon dioxide into the reactor; a reaction furnace which is maintained at a preset temperature and into which the reactor is inserted; a discharge part coupled with the reactor and discharging exhaust gas generated after a reaction of the gas mixture and the sorbents out from the reactor; and analysis part analyzing a carbon dioxide concentration of the exhaust gas discharged from the discharge part, wherein the reactor includes a reactor for wet-type carbon dioxide sorbents and a reactor for dry-type carbon dioxide sorbents, and the reactor for wet-type carbon dioxide sorbents and the reactor for dry-type carbon dioxide sorbents can be alternately inserted into the reaction furnace.

### CROSS-REFERENCE TO RELATED APPLICATIONS

This U.S. non-provisional patent application claims priority under 35 U.S.C. §119 of Korean Patent Application No. 10-2014-0003040, filed on Jan. 9, 2014, the entire contents of which are hereby incorporated by reference.

### BACKGROUND OF THE INVENTION

The present invention disclosed herein relates to an apparatus for testing the performance of carbon dioxide sorbents.

Carbon dioxide is a primary greenhouse gas, which forms the largest portion at 76% or more of greenhouse gases causing global warming, and carbon capture and storage (CCS) technology is emerging as the most realistic and effective alternative capable of directly reducing the amount of carbon dioxide continuously discharged from energy sectors such as power plants.

Furthermore, as the Kyoto Protocol for the Framework Convention on Climate Change is ratified, a carbon dioxide emission trade will be vitalized. Thus, global warming caused by carbon dioxide is emerging as an economic problem and not just an environmental problem.

The cost for carbon dioxide capture forms about 80% of the total cost for capturing, transporting, and storing carbon dioxide, and carbon dioxide sorbents form a great portion of the cost for carbon dioxide capture. Thus, various research has been actively carried out globally.

At present, the most commercialized of sorbents is a liquid chemical sorbent using an amine series solvent, such as monoethanolamine, which shows the highest absorption performance and is therefore used the most.

However, various shortcomings are pointed out for such sorbents, such as their dissipation or deterioration due to evaporation during their capture, their tendency to corrode reactors, and their requiring high levels of energy for their recovery. Thus, in order to solve these various shortcomings, research is actively being done to develop new sorbents, through the injection of additives like piperazine, for example.

Also, research is being carried out on dry-type sorbents such as potassium carbonate, and dry-type adsorbents such as a zeolite, in order to remedy the shortcomings of liquid sorbents, such as wastewater generation, corrosion, and high levels of energy required for recycling.

Korean Patent Application Laid-open Publication No. 2011-0073163 (publicized on Jun. 29, 2011, entitled “Wet-type apparatus and method for separating and recovering carbon dioxide”) discloses a related technology.

### SUMMARY OF THE INVENTION

The present invention provides an apparatus for testing the performance of carbon dioxide sorbents, in which a wet-type carbon dioxide sorbent and a dry-type carbon dioxide sorbent are reacted with a gas mixture containing carbon dioxide under the same conditions so as to evaluate the performance of the sorbents.

Embodiments of the present invention provide an apparatus for testing the performance of carbon dioxide sorbents, the apparatus including: a reactor accommodating carbon dioxide sorbents therein; an injection part coupled to the reactor and injecting a gas mixture containing carbon dioxide into the reactor; a reaction furnace which is maintained at a preset temperature and into which the reactor is inserted; a discharge part coupled to the reactor and discharging exhaust gas, generated after the reaction of the gas mixture and the sorbents, out from the reactor; and analysis part analyzing a carbon dioxide concentration of the exhaust gas discharged from the discharge part, wherein the reactor includes a reactor for wet-type carbon dioxide sorbents and a reactor for dry-type carbon dioxide sorbents, and the reactor for wet-type carbon dioxide sorbents and the reactor for dry-type carbon dioxide sorbents can be alternately inserted into the reaction furnace.

In some embodiments, further included may be an adjustment part which is coupled between the discharge part and the analysis part and adjusts an amount of the exhaust gas discharged from the discharge part.

In other embodiments, the adjustment part may include a back pressure adjustment valve.

In still other embodiments, further included may be a nitrogen supply part connected to the injection part so that the gas mixture may further contain nitrogen and water and which supplies the nitrogen; and a water supply part which is connected to the injection part and supplies the water.

In even other embodiments, further included may be a preheat part connected to the injection part so that a temperature of the gas mixture transferred to the injection part is increased to a preset temperature.

In yet other embodiments, further included may be a water removal part coupled between the adjustment part and the analysis part, and removing water from the exhaust gas discharged from the discharge part.

In further embodiments, the reactor for wet-type carbon dioxide sorbents may include a jet part which is connected to the injection part so that the gas mixture is injected into a liquid sorbent filled in the reactor and which extends to an inner lower portion of the reactor.

In still further embodiments, the jet part may include a bubble filter jetting the gas mixture in the form of micro bubbles to improve reactivity of the gas mixture and the liquid sorbent.

In even further embodiments, further included may be a cooling unit for cooling an internal temperature of the reactor for wet-type carbon dioxide sorbents.

In yet further embodiments, the cooling unit may include a cooling coil in which cooling water flows.

In much further embodiments, further included may be a drain valve formed at a lower portion of the reactor for wet-type carbon dioxide sorbents and discharging the liquid sorbent to the outside.

In still much further embodiments, the reactor for dry-type carbon dioxide sorbents may include a guide part, in which an injection part is connected at an upper portion of the reactor, and which extends to an inner lower portion of the reactor and guides the gas mixture so that a gas mixture injected through the injection part is transferred to the discharge part passing through solid sorbents stacked at an inner lower portion of the reactor.

In even much further embodiments, the guide part may be formed in a cylindrical shape so that one end thereof is coupled to an upper portion of the reactor for dry-type carbon dioxide sorbents, and the other end thereof extends to an inner lower portion of the reactor for dry-type carbon dioxide sorbents.

In yet much further embodiments, the reaction furnace may further include a heat supply part for supplying an amount of heat to maintain a preset temperature thereof.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a view illustrating an apparatus for testing the performance of carbon dioxide sorbents including a reactor for wet-type carbon dioxide sorbents according to an embodiment of the present invention;

FIG. 2 is a view illustrating an apparatus for testing the performance of carbon dioxide sorbents including a reactor for dry-type carbon dioxide sorbents according to an embodiment of the present invention;

FIG. 3 is a view illustrating a reactor for wet-type carbon dioxide sorbents according to an embodiment of the present invention;

FIG. 4 is a view illustrating a gas mixture passing through a reactor for wet-type carbon dioxide sorbents according to an embodiment of the present invention;

FIG. 5 is a view illustrating a reactor for dry-type carbon dioxide sorbents according to an embodiment of the present invention;

FIG. 6 is a view illustrating a gas mixture passing through a reactor for dry-type carbon dioxide sorbents according to an embodiment of the present invention; and

FIG. 7 is a view illustrating a method for evaluating the performance of carbon dioxide sorbents according to an embodiment of the present invention.

### DETAILED DESCRIPTION OF PREFERRED EMBODIMENTS

Since the present invention may have diverse modified embodiments, preferred embodiments are illustrated in the drawings and are described in the detailed description of the invention. However, this does not limit the present invention within specific embodiments and it should be understood that the present invention covers all the modifications, equivalents, and replacements within the idea and technical scope of the present invention. Moreover, detailed descriptions related to well-known functions or configurations will not be included in order not to unnecessarily obscure subject matters of the present invention.

It will be understood that although the terms ‘first’ and ‘second’ are used herein to describe various elements, these elements should not be limited by these terms. The terms are only used to distinguish one component from other components.

In the following description, the technical terms are used only for explaining a specific exemplary embodiment while not limiting the present invention. The terms used in the singular form may include the plural form unless otherwise stated. The meaning of ‘include’ or ‘comprise’ specifies a property, a region, a fixed number, a step, a process, an element and/or a component but does not exclude other properties, regions, fixed numbers, steps, processes, elements and/or components.

Hereinafter, embodiments of the present invention are described in more detail with reference to the accompanying drawings. In the description of the accompanying drawings, same or corresponding components are given the same reference numbers in the drawings, and overlapping descriptions thereof will not be provided.

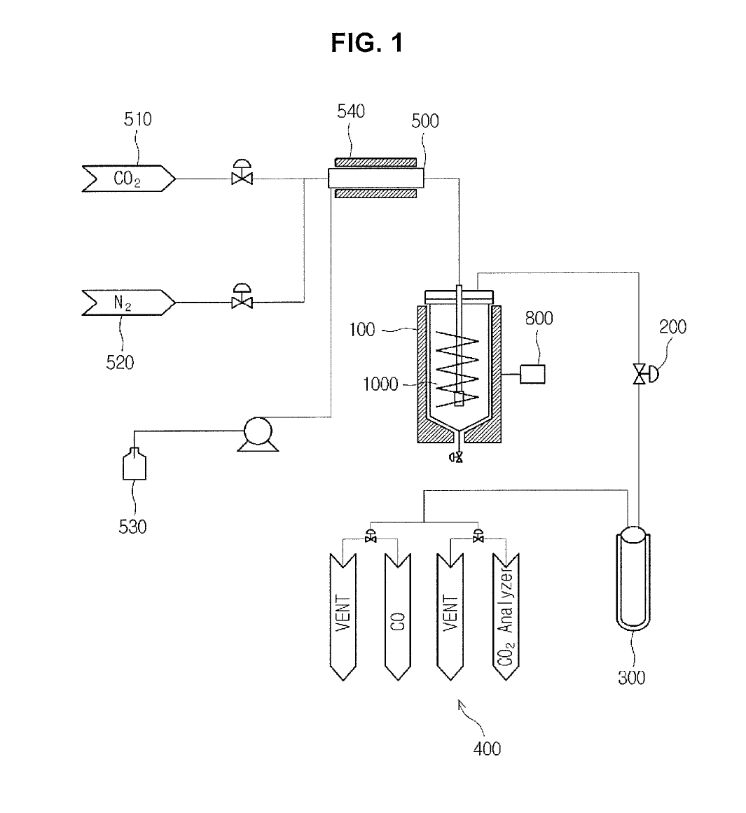

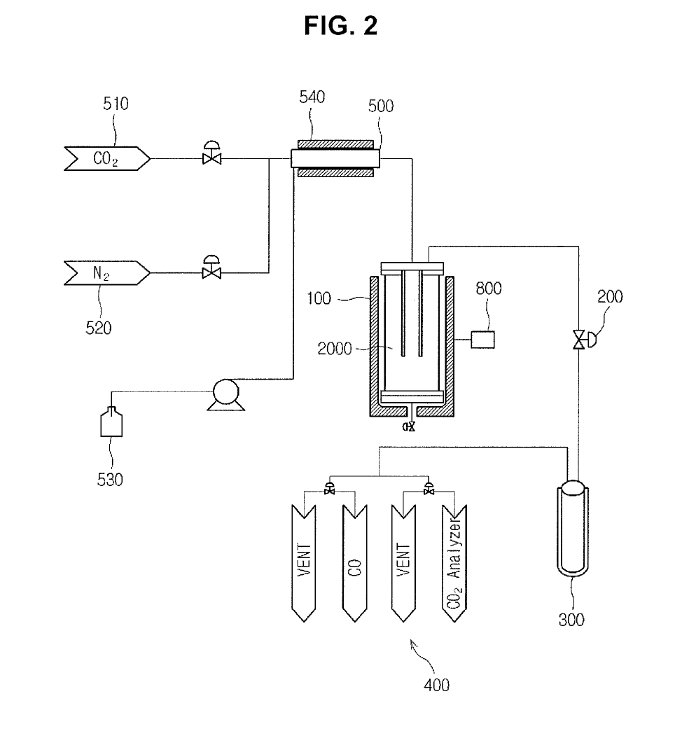

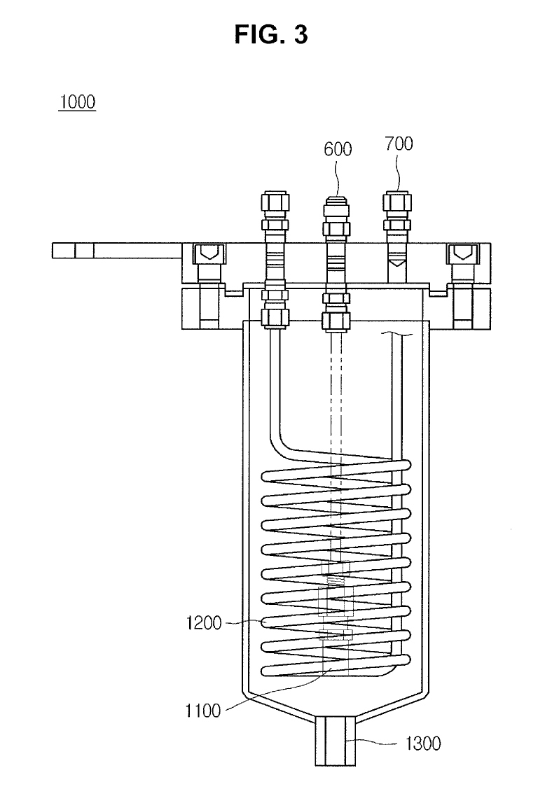

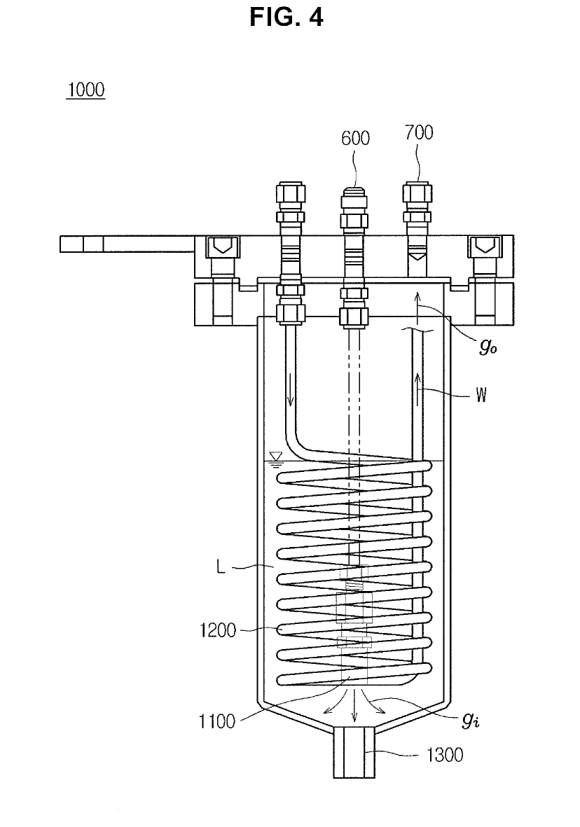

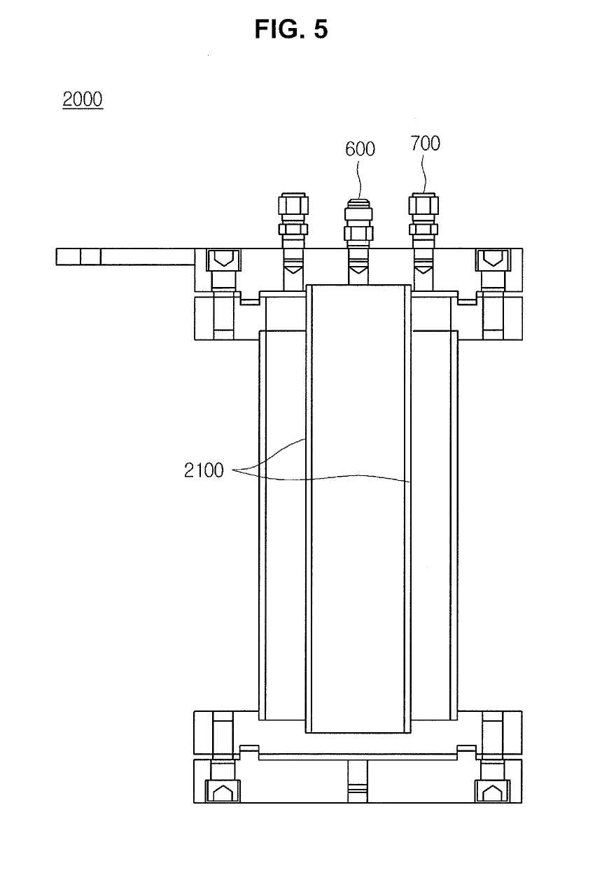

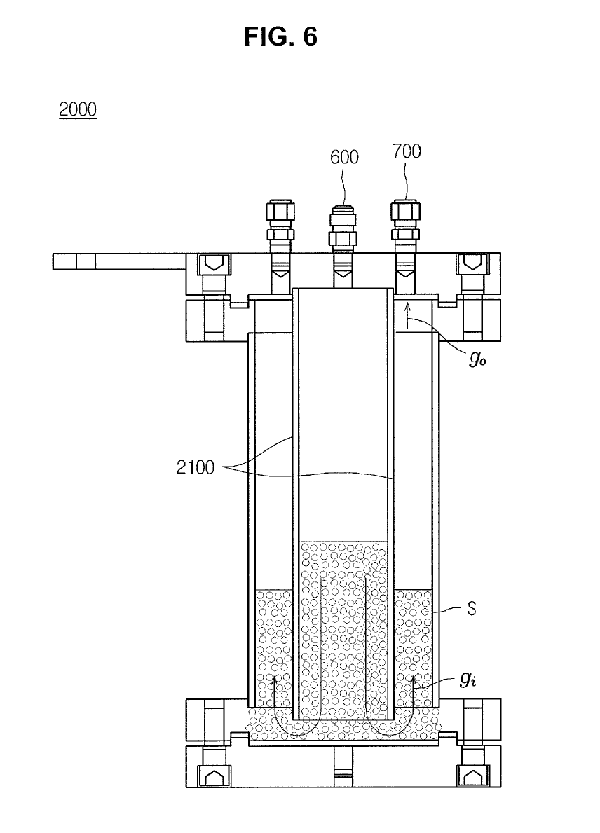

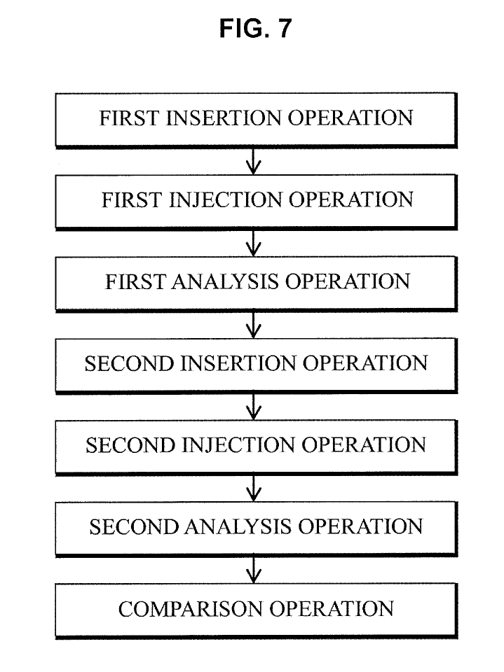

FIG. 1 is a view illustrating an apparatus for testing the performance of carbon dioxide sorbents including a reactor for wet-type carbon dioxide sorbents according to an embodiment of the present invention, FIG. 2 is a view illustrating an apparatus for testing the performance of carbon dioxide sorbents including a reactor for dry-type carbon dioxide sorbents according to an embodiment of the present invention, FIG. 3 is a view illustrating a reactor for wet-type carbon dioxide sorbents according to an embodiment of the present invention, FIG. 4 is a view illustrating a gas mixture passing through a reactor for wet-type carbon dioxide sorbents according to an embodiment of the present invention, FIG. 5 is a view illustrating a reactor for dry-type carbon dioxide sorbents according to an embodiment of the present invention, FIG. 6 is a view illustrating a gas mixture passing through a reactor for dry-type carbon dioxide sorbents according to an embodiment of the present invention, and FIG. 7 is a view illustrating a method for evaluating the performance of carbon dioxide sorbents according to an embodiment of the present invention.

An object of the present invention is to compare the performance of each sorbent by reacting dry-type carbon dioxide sorbents S and wet-type carbon dioxide sorbents with a gas mixture gi containing carbon dioxide under the same conditions. As can be seen with reference to FIGS. 1 and 2, a reaction of carbon dioxide and sorbents progresses while only a reactor for dry-type carbon dioxide sorbents 2000 and a reactor for wet-type carbon dioxide sorbents 1000 is alternately used, and the remaining conditions are set to be the same.

As illustrated in FIG. 7, in order to evaluate the performance of the wet and dry-type carbon dioxide sorbents S, firstly, a first insertion operation for inserting the reactor 2000 for dry-type carbon dioxide sorbents into the reaction furnace 100, a first injection operation for injecting a gas mixture gi containing carbon dioxide into the reactor 2000 for dry-type carbon dioxide sorbents, and a first analysis operation for analyzing a carbon dioxide concentration from exhaust gas go generated after a reaction of the dry-type carbon dioxide sorbents S and the gas mixture gi may be sequentially performed. Next, a reactor 1000 for wet-type carbon dioxide sorbents is inserted into the same reaction furnace 100 instead of the reactor 2000 for dry-type carbon dioxide sorbents, and then the same procedure may be repeated.

That is, a second insertion operation for inserting the reactor 2000 for wet-type carbon dioxide sorbents into the reaction furnace 100, a second injection operation for injecting a gas mixture gi containing carbon dioxide into the reactor 2000 for wet-type carbon dioxide sorbents, and a second analysis operation for analyzing a carbon dioxide concentration from exhaust gas go generated after a reaction of the wet-type carbon dioxide sorbents L and the gas mixture gi may be sequentially performed.

As described above, carbon dioxide concentrations analyzed through the first and second analysis operations are compared, so that the performance of the dry-type carbon dioxide sorbents S and the wet-type carbon dioxide sorbent L may be evaluated. Here, it is obvious that performance of the wet-type carbon dioxide sorbents L is firstly tested, then the reactor is replaced, and then the performance of the dry-type carbon dioxide sorbents S may be tested.

Here, the reactor 100 is set to be maintained at the same temperature, and the pressure in the reactor is also maintained at a uniform same pressure. That is, the temperature of the reaction furnace 100 is maintained at a preset temperature, so that the amount of heat transmitted to the reactor 1000 for wet-type carbon dioxide sorbents and the reactor 2000 for dry-type carbon dioxide sorbents, which are inserted into the reaction furnace 100, may also become the same. Also, the amount of exhaust gas go discharged from each reactor is adjusted, so that the pressure during a reaction in each reactor may be adjusted to be the same.

Thus, each of the sorbents and gas mixtures are reacted inside the reactor 1000 for wet-type carbon dioxide sorbents and the reactor 2000 for dry-type carbon dioxide sorbents under the same temperature and pressure conditions, so that the performance of the dry-type carbon dioxide sorbents S and the wet-type carbon dioxide sorbents L may be compared.

To this end, an apparatus for testing the performance of carbon dioxide sorbents according to an embodiment of the present invention, may include a reactor, an injection part 600, a reaction roll, a discharge part 700, and an analysis part 400, wherein the reactor may include a reactor 1000 for wet-type carbon dioxide sorbents and a reactor 2000 for dry-type carbon dioxide sorbents.

The reactor includes sorbents for absorbing carbon dioxide therein, and the injection part 600 coupled to the reactor injects a gas mixture gi containing carbon dioxide into the reactor. Here, the injection part 600 is formed on an upper portion of the reactor and injects the gas mixture gi from the upper portion to a lower portion of the reactor. The sorbents and the gas mixture meet and react with each other inside the reactor, and the carbon dioxide sorbents capture carbon dioxide.

Here, a heat supply part 800 for supplying an amount of heat to the reaction furnace 100 may be coupled to the reaction furnace 100 to maintain a preset temperature of the reaction furnace 100. The heat supply part 800 checks the temperature of the reaction furnace 100 and adjusts the amount of heat supplied to the reaction furnace 100 to maintain a preset temperature of the reaction furnace 100. That is, when the temperature of the reaction furnace 100 is less than a preset temperature, the temperature of the reaction furnace 100 is elevated by increasing the amount of heat supplied to the reaction furnace 100, and when the temperature of the reaction furnace 100 is more than a preset temperature, the temperature of the reaction furnace 100 is lowered by decreasing or removing the amount of heat supplied to the reaction furnace 100.

The discharge part 700 may discharge exhaust gas go, which is generated after a reaction of the carbon dioxide sorbents and the gas mixture inside the reactor, out from the reactor. Here, the discharge part 700 is formed at the upper portion of the reactor and discharges the exhaust gas go after a reaction. Since the injection part 600 and the discharge part 700 are formed together at the upper portion of the reactor, a reactor 1000 for wet-type carbon dioxide sorbents may include an extension portion and a reactor 2000 for dry-type carbon dioxide sorbents may include a guide portion 2100, respectively, so that the gas mixture gi injected from the injection part 600 may not be directly discharged to the discharge part 700.

The analysis part 400 is connected to the discharge part 700 and may analyze a carbon dioxide concentration of the exhaust gas go discharged from the discharge part 700. Here, the analysis part 400 may include a non-dispersive infrared (NDIR) CO2 analyzer which analyzes the carbon dioxide concentration in air by using a non-dispersion infrared sensor.

As described above, an object of the present invention is to compare the performance of each sorbent by reacting wet-type carbon dioxide sorbents L and dry-type carbon dioxide sorbents S with a gas mixture gi, which contains carbon dioxide, under the same conditions, and analyzing the concentration of discharged gas. Accordingly, the same injection part 600, the same discharge part 700, the same reaction furnace 100 maintaining a preset temperature, the same analysis part 400, and the same reaction furnace 100 are used. However, only a reactor 1000 for wet-type carbon dioxide sorbents and a reactor 2000 for dry-type carbon dioxide sorbents are used by alternately being inserted into the reaction furnace 100.

Also, an adjustment part 200 may be further included between the discharge part 700 and the analysis part 400 to maintain the reactor at a uniform pressure. The adjustment part 200 includes a back pressure adjustment valve. The back pressure adjustment valve is an adjustment valve discharging fluid according to a pressure change at a first side in order to maintain the fluid pressure at the first side at a preset pressure, and adjusts the amount of exhaust gas go according to a pressure change inside the reactor to maintain a predetermined fluid pressure in the reactor.

The temperature of the reaction furnace 100 into which the reactors are inserted is maintained at a preset temperature, and the pressure of each reactor during a reaction is adjusted to be uniform, so that performance of the wet-type carbon dioxide sorbents L and the dry-type carbon dioxide sorbents S may be evaluated under the same conditions.

Here, the included amount of nitrogen and water may be increased so that the gas mixture gi may be similar to real flue gas. That is, a nitrogen supply part 520 and a water supply part 530 may be further included in addition to a carbon dioxide supply part 510, so that a gas mixture gi containing carbon dioxide, nitrogen, and water may be generated. The carbon dioxide, the nitrogen, and the water supplied from each supply part may be mixed in a mixing part 500.

Also, an embodiment of the present invention may include a preheat part 540 for increasing a temperature of the gas mixture gi up to a preset temperature so that the gas mixture gi mixed at the mixing part 500 may simulate real flue gas. The preheat part 540 may increase the temperature of the gas mixture gi mixed inside the mixing part 500 up to a preset temperature by heating the mixing part 500.

Thus, the gas mixture gi prepared by mixing nitrogen, water, and carbon dioxide supplied from each supply part, is heated to a preset temperature to be injected into each reactor. That is, gas mixtures gi prepared under the same conditions are injected into the reactor 1000 for wet-type carbon dioxide sorbents and a reactor 2000 for dry-type carbon dioxide sorbents, respectively, and reactions are allowed to progress. FIG. 1 illustrates an apparatus for analyzing the performance of absorbing carbon dioxide of the wet-type carbon dioxide sorbents L by using the reactor 1000 for wet-type carbon dioxide sorbents, and FIG. 2 illustrates an apparatus for analyzing the performance of absorbing carbon dioxide of the dry-type carbon dioxide sorbents S by using the reactor 2000 for the wet-type carbon dioxide sorbents

Referring to FIGS. 3 and 4, the reactor 1000 for wet-type carbon dioxide sorbents will be described below. As illustrated in FIGS. 3 and 4, the reactor 1000 for wet-type carbon dioxide sorbents includes a jet part 1100 which is connected to an injection part 600 so that a gas mixture gi may be injected to liquid sorbent filled in the reactor, and which extends to an inner lower portion of the reactor. That is, the injection part 600 is formed at an upper portion of the reactor, and one end of the jet part 1100 is connected to the injection part 600 while the other end extends to a lower portion of the reactor, so that the gas mixture gi injected through the injection part 600 may move to a lower portion of the reactor. Here, the liquid sorbent is filled in the reactor while maintaining a certain liquid level. The gas mixture gi injected through the injection part 600 is reacted while passing through the liquid sorbent. A predetermined amount of carbon dioxide in the gas mixture gi is absorbed by the liquid sorbents, and exhaust gas go, which is not absorbed, is discharged to the outside through a discharge part 700. Here, the discharge part 700 is connected to an analysis part 400. That is, a predetermined amount of carbon dioxide in the gas mixture gi is absorbed by the liquid sorbent, and the exhaust gas go, which is not absorbed, is discharged to the outside through the discharge part 700 to be transferred to the analysis part 400. Thus, the amount of carbon dioxide of the exhaust gas go is transferred to the analysis part 400, so that the performance of the liquid sorbent may be evaluated. Here, the temperature of the reactor may be set to 100° C. or less.

Also, when the temperature of the reactor is increased to 120° C. or more, carbon dioxide absorbed by the liquid sorbent may be separated from the liquid sorbent. The separated carbon dioxide and exhaust gas go containing the separated carbon dioxide are transferred to the analysis part 400 through the discharge part 700, so that a carbon dioxide concentration in the exhaust gas go may be measured. Thus, the performance of absorbing carbon dioxide of the liquid sorbent may be once again confirmed.

Here, the jet part 1100 may include a bubble filter jetting the gas mixture gi in the form of micro bubbles to improve the reactivity of the gas mixture gi and the liquid sorbent. That is, a jetted mixture gi is formed in a small enough size so that the jetted gas mixture gi may be reacted with the liquid sorbent as much as possible.

Also, an apparatus for testing the performance of carbon dioxide sorbents according to an embodiment of the present invention, may further include a cooling unit 1200 for cooling the internal temperature of the reactor 1000 for wet-type carbon dioxide sorbents. Since a carbon dioxide absorption reaction is an exothermic reaction, the internal temperature of the reactor may be increased by the exothermic reaction. To adjust this, the cooling unit 1200 for cooling the internal temperature of the reactor may be further included.

Here, the cooling unit 1200 may include a cooling coil in which cooling water W flows. The cooling coil is preferably formed to a degree so as to be sufficiently dipped into the liquid sorbent. The cooling water W flows in the cooling coil, and cancels out heat generated by the carbon dioxide absorption reaction, so that the internal temperature of the reactor may also be maintained at a predetermined temperature.

Also, of the reactor 1000 for wet-type carbon dioxide sorbents may further include a drain valve 1300 for discharging the liquid sorbent at a lower portion thereof. That is, the drain valve allows the liquid sorbent to be readily discharged to the outside after a reaction.

Referring to FIGS. 5 and 6, the reactor 2000 for dry-type carbon dioxide sorbents will be described below. As illustrated in FIGS. 5 and 6, the reactor 2000 for dry-type carbon dioxide sorbents has an upper portion connected to an injection part 600, and includes a guide part 2100, which extends to an inner lower portion of the reactor and guides the gas mixture gi so that a gas mixture gi injected through the injection part 600 may be transferred to the discharge part 700 passing through solid sorbents stacked at an inner lower portion of the reactor. That is, the guide part 2100 is further included so that the gas mixture gi injected through the injection part 600 may not be directly transferred to discharge gas go, but may be transferred after passing through the solid sorbents.

The guide part 2100 spatially separates the injection part 600 and the discharge part 700, so that the gas mixture gi is prevented from directly moving from the injection part 600 to the discharge part 700. That is, the guide part 2100 is formed in a cylindrical shape to surround the injection part 600, and has one end coupled to an upper portion of the reactor 2000 for dry-type carbon dioxide sorbents, and the other end extending to an inner lower portion of the reactor 2000 for dry-type carbon dioxide sorbents. Accordingly, the gas mixture gi injected through the injection part 600 moves to a lower side of the reactor along the inside of the guide part 2100, passes through the solid sorbents stacked at a lower portion of the reactor, and moves to the discharge part 700.

The gas mixture gi injected from the injection part 600 is reacted while passing through the solid sorbents. A predetermined amount of carbon dioxide in the gas mixture gi is absorbed by the solid sorbents, and exhaust gas go, which is not absorbed, is discharged to the outside through the discharge part 700. A predetermined amount of carbon dioxide in the gas mixture gi is absorbed by the sorbents, and the remaining exhaust gas go is discharged to the discharge part 700 and is transferred to the analysis part 400. Thus, the amount of carbon dioxide of the exhaust gas go transferred to the analysis part 400 is measured, so that the performance of the solid sorbents may be evaluated.

Here, the temperature of the reactor 2000 for dry-type carbon dioxide sorbents may be set to the same temperature of 100° C. or less as the reactor 1000 for wet-type carbon dioxide sorbents.

Also, when the temperature of the reactor 2000 for dry-type carbon dioxide sorbents is increased to 120° C. or more, carbon dioxide absorbed by the solid sorbents may be separated from the solid sorbents. The separated carbon dioxide and the exhaust gas go containing the separated carbon dioxide are transferred to the analysis part 400 through the discharge part 700, so that a carbon dioxide concentration in exhaust gas go may be measured. Thus, the performance of carbon dioxide absorption by the solid sorbents may be once again confirmed.

Referring to FIGS. 1 and 2, an apparatus for testing the performance of carbon dioxide sorbents according to an embodiment of the present invention, may further include a water removal part 300 coupled between the adjustment part 200 and the analysis part 400 and removing water from the exhaust gas go discharged from the discharge part 700. That is, water contained in the exhaust gas go is removed before the exhaust gas go discharged form the discharge part 700 moves to the analysis part, so that an analysis of the carbon dioxide concentration may be more readily and accurately performed.

Here, the water removal part 300 may include a chiller maintaining the temperature of the water removal part 300 at a low temperature (about 10° C. or less), and a separator liquefying vaporized water and storing the water.

According to embodiments of the present invention, wet-type carbon dioxide sorbents and dry-type carbon dioxide sorbents are reacted with a gas mixture containing carbon dioxide under the same conditions, so that the performance of the sorbents can be evaluated by using an apparatus for testing the performance of carbon dioxide sorbents.

Although the invention has been described with reference to particular embodiments, this description is not meant to be construed in a limiting sense. Various modifications of the disclosed embodiments as well as alternative embodiments of the invention through additions, changes, deletions, or supplements of components without departing from the scope of the invention will become apparent to persons skilled in the art. It is therefore deemed that the appended claims will cover any such modifications or embodiments that fall within the scope of the invention.

What is claimed is:

1. An apparatus for testing performance of carbon dioxide sorbents, the apparatus comprising:
a reactor accommodating carbon dioxide sorbents therein;
an injection part coupled to the reactor and injecting a gas mixture containing carbon dioxide into the reactor;
a reaction furnace which is maintained at a preset temperature and into which the reactor is inserted;
a discharge part coupled to the reactor and discharging exhaust gas, which is generated after a reaction of the gas mixture and the sorbents, out from the reactor; and
an analysis part analyzing a carbon dioxide concentration of the exhaust gas discharged from the discharge part, wherein
the reactor comprises a reactor for wet-type carbon dioxide sorbents and a reactor for dry-type carbon dioxide sorbents, and the reactor for wet-type carbon dioxide sorbents and the reactor for dry-type carbon dioxide sorbents can be alternately inserted into the reaction furnace.

2. The apparatus of claim 1, further comprising an adjustment part coupled between the discharge part and the analysis part and adjusting an amount of the exhaust gas discharged from the discharge part.

3. The apparatus of claim 2, wherein the adjustment part comprises a back pressure adjustment valve.

4. The apparatus of claim 2, further comprising:
a nitrogen supply part, which is connected to the injection part so that the gas mixture may further contain nitrogen and water, supplies the nitrogen; and
a water supply part, which is connected to the injection part, supplies the water.

5. The apparatus of claim 2, further comprising a preheat part connected to the injection part such that a temperature of the gas mixture transferred to the injection part is increased to a preset temperature.

6. The apparatus of claim 2, further comprising a water removal part coupled between the adjustment part and the analysis part, and removing water from the exhaust gas discharged from the discharge part.

7. The apparatus of claim 2, wherein the reactor for wet-type carbon dioxide sorbents further comprises a jet part connected to the injection part such that the gas mixture is injected into liquid sorbent filled in the reactor, and extending to an inner lower portion of the reactor.

8. The apparatus of claim 7, wherein the jet part comprises a bubble filter jetting the gas mixture in the form of micro bubbles to improve reactivity of the gas mixture and the liquid sorbent.

9. The apparatus of claim 7, further comprising a cooling unit for lowering an internal temperature of the reactor for wet-type carbon dioxide sorbents.

10. The apparatus of claim 9, wherein the cooling unit comprises a cooling coil in which cooling water flows.

11. The apparatus of claim 7, further comprising a drain valve disposed at a lower portion of the reactor for wet-type carbon dioxide sorbents and discharging the liquid sorbent to the outside.

12. The apparatus of claim 2, wherein the reactor for dry-type carbon dioxide sorbents has an upper portion connected to the injection part, and comprises a guide part which extends to an inner lower portion of the reactor and guides the gas mixture so that a gas mixture injected through the injection part is transferred to the discharge part after passing through solid sorbents stacked at an inner lower portion of the reactor.

13. The apparatus of claim 12, wherein the guide part is formed in a cylindrical shape so that one end thereof is coupled to an upper portion of the reactor for dry-type carbon dioxide sorbents, and the other end thereof extends to an inner lower portion of the reactor for dry-type carbon dioxide sorbents.

14. The apparatus of claim 2, wherein the reaction furnace further comprises a heat supply part for supplying an amount of heat to maintain a preset temperature thereof.
